(19) United States
(12) Patent Application Publication
(10) Pub. No.: US 2009/0018436 A1
(43) Pub. Date: Jan. 15, 2009

(54) OPTICAL COHERENCE TOMOGRAPHY

(76) Inventors: Daniel Gey Van Pittius, Stafford (GB); Monica Spiteri, Staffordshire (GB); Suzanne Claire Whiteman, Staffordshire (GB)

Correspondence Address: TREGO, HINES & LADENHEIM, PLLC, 9300 HARRIS CORNERS PARKWAY, SUITE 210, CHARLOTTE, NC, 28269-3797, US

(21) Appl. No.: 11/997,237

(22) PCT Filed: Jul. 14, 2006
(86) PCT No.: PCT/GB2006/002616

§ 371 Date: Sep. 9, 2008

(30) Foreign Application Priority Data
Jul. 30, 2005 (GB) 0515758.1

(51) Int. Cl. A61B 5/05

(52) U.S. Cl. 600/425

(57) ABSTRACT

A method of determining a disease state of an animal by imaging a sample of at least a portion of a tissue of the animal by optical coherence tomography scanning and comparing optical coherence tomographical scanned image the histopathological images of a tissue type of the animal at various stages of disease state stored in a histopathological image database.

### FIELD OF THE INVENTION

This invention relates to optical coherence tomography, and in particular but not exclusively to methods of determining disease states, and a kit of parts.

### BACKGROUND TO THE INVENTION

Recent advances in biomedical imaging technologies have led to increased diagnostic challenges for the early detection of various cancers. These include continual developments in low-dose spiral computed tomography; positron emission tomography (PET) and magnetic resonance imaging. However, whilst these technologies are significantly better than conventional diagnostic approaches, they require significant costly infrastructural changes to accommodate them and are thus limited in availability due to their requirement/upkeep costs. In addition, low-dose computed tomography screening has been implicated in simultaneous increased malignancy risk per se, due to higher radiation levels compared with conventional radiography.

Current imaging techniques employed for the diagnosis of some diseases, and lung disease in particular, do not provide sufficient resolution to detect critical early pathological changes within certain anatomical tissues such as the bronchial epithelium. Lung cancer is the most common malignancy in the western world at the time of writing, and is the leading cause of cancer-related deaths. It is recognised that over 85% of lung tumours originate within the bronchial epithelium, with multi-stage cellular changes progressing over a relatively long period of time prior to first presentation of invasive cancer. Thus as the lesion develops and progresses, the in situ microstructural profile of the bronchial epithelium slowly changes. It follows that programmes aimed at decreasing lung cancer morbidity/mortality and enhancing screening procedures, need to invest in development of safe, reliable diagnostic techniques capable of detecting in situ micro-invasive pathological lesions, potentially at relatively curable stages. Additionally such cost-effective tools would aim to be consumer—as well as environmentally—friendly. The availability of such an advanced optical tool would provide a tremendous improvement in lung cancer is surveillance.

Direct airway imaging has involved fluorescence bronchoscopy, which enhances the identification and diagnosis of in situ mucosal abnormalities such as early cancerous changes. However, auto-fluorescence bronchoscopy (LIFE-bronchoscopy) has limitations in terms of resolution and penetration of tissue depth. Other bronchoscopic technologies encompassing incorporation of high-frequency ultra-sonography such as EBUS (endobronchial ultrasonography), do achieve deep penetration of the bronchial airway tissue, but their spatial resolution is insufficient for clear demarcation of the multi-layered bronchial epithelium and identification of in situ morphological changes. In addition, due to small differences of acoustic impedance between the normal and malignant tissues, the imaging contrast offered by EBUS is inherently low.

A relatively new imaging technology capable of producing rapid, high-resolution cross-sectional images of biological tissues in a non-invasive fashion is optical coherence tomography (hereinafter “OCT”). As such, OCT represents a technological shift from the above-mentioned methods, but is analogous to B-mode ultra-sonography; but rather than using a sound signal, it utilises light. OCT delivers infrared light waves to the image site through a single-optical fibre; the light reflects off the internal micro-structural layers within the scanned tissue, allowing micron-scale resolution pick-up of the normal anatomy and any in situ morphological aberrations. Signal processing involves low coherence interferometry, which is the analysis of reflected light waves from the internal tissue micro-structures (Huang D, Swanson E A, Lin C P, et al. Optical Coherence Tomography, Science 1991; 254: 1178-1181; and Fujimoto J G, Boppart S A, Tearney G J, et al. High Resolution in vivo Intra-arterial Imaging with Optical Coherence Tomography. Heart 1999; 82: 128-133). The coherence length of the light source determines the longitudinal resolution. With the appropriate light source, OCT imaging can reliably produce a 5-15 μm resolution, as compared to 150 μm for high-frequency ultrasonography. Over the past ten years, the clinical use of OCT imaging has been tested in a variety of biological tissues, ex vivo, including retinal tissue, skin, gastro-intestinal tract, urologic tissues, cartilage, tendon and tooth.

In contrast, research on the feasibility of OCT for imaging of the human respiratory system is comparatively sparse (Pitris C, Brezinski M E, Bouma B E, et al. High Resolution Imaging of Upper Respiratory Tract with Optical Coherence Tomography: A Feasibility Study. Am J Respir Crit Care Med 1998: 157: 1640-1644; and Yang Y, Whiteman S C, Gey van Pittius D, et al. Use of Optical Coherence Tomography in Delineating Airways Micro-structure: Comparison of OCT Images to Histo-pathological Sections. Phys. Med. Biol. 2004; 49: 1247-1255).

It would be advantageous to provide OCT imaging in real time on sections of fresh human lung tissue, especially from patients undergoing lung resection surgery, and explore its capability to identify the micro-structural components of the bronchial epithelium. It would furthermore be advantageous to detect in situ morphological/pathological aberrations in lung tissue and other tissues.

It would also be advantageous to provide an apparatus for performing OCT in situ, with additional means for a physician to correlate the OCT images obtained, in real time and accurately, with known images of a target tissue type in various stages of a disease state.

It is therefore an aim of preferred embodiments of the present invention to overcome or mitigate at least one problem in the prior art, whether expressly disclosed herein or not.

### SUMMARY OF THE INVENTION

According to a first aspect of the invention there is provided a method of determining a disease state of an animal, a method comprising the steps of:
(a) providing a plurality of histopathological images of a tissue type of an animal at various stages of a disease state;
(b) imaging a sample of at least a portion of the same tissue type of a target animal by optical coherence tomography scanning; and
(c) comparing the optical coherence tomographical scanned image with the histopathological images of the tissue to determine the disease state of the animal.

Preferably step (a) comprises providing histopathological images of a tissue type of an animal at various stages of a cancerous state. Preferably, step (a) comprises providing histopathological images of a tissue type at least in a non-cancerous, pre-cancerous, inflamed and cancerous state. Step (a) may comprise providing a plurality of histopathological images of a tissue type for each of the non-cancerous, pre-cancerous, inflamed and cancerous states.

Thus, step (a) may comprise for example providing a plurality of histopathological images of a tissue type in a non-cancerous state, and/or providing a plurality of histopathological images of a tissue type in a pre-cancerous state, each of the plurality of pre-cancerous images displaying a different stage of the pre-cancerous stage; and/or a plurality of histopathological images of the inflamed state of a tissue type, each image showing a different stage of the inflamed state; and/or a plurality of histopathological images of tissue in the cancerous stage, each image displaying the tissue type in a different stage of the cancerous state.

In preferred embodiments, there are provided a plurality of histopathological images of a tissue type for each of the non-cancerous, pre-cancerous, inflamed and cancerous states, showing variations within specific structures in the tissue type for each state. For example, the plurality of images in any state may show varying structures of connective tissue, and/or the plurality of images of the cancerous state may show cancerous cells at various stages of growth and penetration into the tissue or into structures within the tissue.

The tissue type may be any suitable tissue type from the animal. The tissue type is preferably lung tissue. The animal is preferably a mammal, and more preferably a human.

Imaging of the tissue sample in step (b) by optical coherence tomography preferably comprises optical coherence tomography scanning using a Michelson-type interferometer.

Preferably the method comprises a method of determining a disease stage in situ of an animal. Suitably, the method comprises in step (b) of imaging a sample in situ of a tissue type of the target animal by optical coherence tomography scanning.

Alternatively or additionally, imaging of the sample of tissue of the target animal in step (b) by optical coherence tomography scanning may comprise removing a sample of the tissue from the target animal and imaging at least a portion of the removed tissue remote from the animal.

Step (c) may comprise comparing a single optical coherence tomogram image with one or more of the plurality of histopathological images. Step (c) may comprise comparing a single optical coherence tomogram image with all of the histopathological images, to determine which of the histopathological images substantially correlates with the optical coherence tomogram. Alternatively or additionally, step (c) may comprise comparing a plurality of optical coherence tomogram images of the tissue of the target animal with one or more of the plurality of histopathological images. For example, step (c) in one embodiment may comprise comparing a single optical coherence tomogram image of, for example, cancerous tissue, with one or more of a plurality of histopathological images of the same tissue in a cancerous state, or may comprise comparing the optical coherence tomogram image with all of the plurality of histopathological images to determine which histopathological image most readily corresponds to the optical coherence tomogram.

The histopathological images may be manufactured by taking an image of sections of a relevant tissue at various stages of a disease state. The images may be for example photographs, electron micrographs, or the like. Preferably the image is a cross-sectional view of the tissue, showing depth of the tissue. Preferably the optical coherence tomogram images obtained in step (b) substantially correspond in image dimensions and angle to the cross-sectional histopathological images of step (a).

The histopathological images are preferably stored in a database, more preferably an electronic database.

Suitably the or each OCT image is compared in step (c) with the database of histopathological images. Suitably the or each OCT image is stored on a database, more preferably an electronic database, and preferably step (c) comprises comparison of the OCT image database with a histopathological image database. Suitably the OCT image database and the histopathological image database are both electronic databases and step (c) comprises electronic comparison of the databases by electrical computing means such as an electronic computer, for example.

Suitably the means to electronically compare the database of histopathological images with the database of OCT images comprises means to indicate when a potential match occurs between a histopathological image. Thus the means to electronically compare the histopathological image database and OCT image database may preferably indicate to a user when, for example, a particular tissue state such as a tumour or cancer cells, is present in an OCT image of a tissue sample by comparing it with histopathological image of the same tissue type having the same known tissue structure such as the tumour or cancer cells, for example.

Preferably the method further comprises a step of generating a database of optical properties of the tissue type or microstructures of the tissue type. This step may be performed at any point in the method; before step (a), between steps (a) and (b), between steps (b) and (c), or after step (c). Preferably the method further comprises comparing the optical coherence tomographical scanned image with the database of optical properties of the tissue, to determine properties of the tissue scanned in the OCT image.

Generation of a database of optical properties may be performed by imaging a tissue type using a spectrophotometer. Optical properties included in the database may include the transmission, diffuse reflectance, scattering and absorption properties of a tissue type. Suitably the spectrophotometer is used to image a tissue type over a wavelength range of 500 nm to 2200 nm, more preferably 600 nm to 2000 nm.

The database of optical properties is preferably an electronic database, and more preferably associated with an electronic computer. Preferably the method comprises a step of comparing the optical coherence tomographical scanned image with the database of optical properties electronically, and may include a further step of indicating to a user when a match of one or more optical properties from the database of optical properties of the tissue type occurs with an OCT image.

According to a second aspect of the invention there is provided a kit comprising
(a) an optical coherence tomography apparatus comprising an interferometer comprising a light source, a beam splitter, a reference arm comprising a reference mirror, and a sample arm comprising a sample probe through which light emitted from the broadband light source may pass, and a means to analyse light reflected from the reference arm and sample probe; and
(b) a library of histopathological images of a tissue type of an animal at various stages of a disease state.

Preferably the histopathological images, the tissue type, the animal and the disease state are as described hereinabove for the first aspect of the invention.

Preferably the interferometer is a Michelson-type interferometer.

Preferably the broadband light source has a central wavelength of between 1000 nm and 1500 nm, more preferably between 1100 nm and 1400 nm, most preferably substantially 1300 nm. Preferably the broadband light source has a band width of between 40 nm and 60 nm, more preferably between 45 nm and 55 nm, and most preferably between 50 nm and 53 nm.

Preferably substantially 50% of the light emitted by the broadband light source is directed to the reference arm.

Preferably the reference arm comprises a double pass scanning apparatus, preferably comprising a collimator, neutral density filter, grating, double pass mirror, optical lens and reflecting mirror. Preferably the interferometer comprises fibre optics through which light from the broadband light source travels. Preferably the probe head of the sample arm comprises a portable body through which a fibre optic cable terminates, the fibre optic cable being connected to the broadband light source, preferably by way of a 50/50 optical fibre coupler (which splits a single optical fibre from the broadband light source into two optical fibres, one of which goes to the reference arm, and one of which goes to the probe head).

The probe head preferably comprises a collimating lens, and an objective lens. Thus the collimating lens and objective lens are capable of focussing the light onto the target tissue and enabling back-scattered light from the tissue to be analysed by the means to analyse light reflected from the probe.

In the interferometer, the 50% of the light directed towards the sample through the probe head is back-scattered through the optical fibre and combined with light reflected from the reference arm. Polarisation controllers may be utilised to achieve the maximum obtainable interference fringe visibility. The interferometer may also comprise a balanced detector scheme to minimise noise from the light source. Suitably the means to analyse the light reflected from the reference arm and sample probe comprises electronic means, such as an electronic computer. The electronic means may include an amplifier filter, demodulator or any mixture thereof.

The apparatus may further comprise an electronic computer and/or a display means. The display means may comprise a display screen such as a computer monitor, television or the like, for example.

Preferably the electronic computer comprises a database, capable of storing the OCT images produced by the interferometer. Suitably the electronic computer comprises means to compare an OCT image with histopathological images contained in the library of histopathological images. Preferably the electronic computer comprises means to indicate when a potential match between an OCT image and a histopathological image from the image library occurs. For example, a user may wish to know whether a particular OCT image of a target tissue comprises the same structures as a known histopathological image, e.g. cancer cells, a tumour, a particular connective tissue morphology, and the like.

Preferably the library of histopathological images of a tissue type of an animal at various stages of a disease state comprises a plurality of histopathological images of a specific tissue type at various stages of a disease state. Preferably the plurality of histopathological images comprises images of a specific tissue at various stages of a cancerous state, more preferably at least one image of the tissue in a healthy state, at least one image of the tissue in a pre-cancerous state, at least one image of the tissue in an inflamed state and at least one image of the tissue in a cancerous state.

The library of histopathological images is preferably a database of images and more preferably an electronic database.

Preferably the kit further comprises a database of optical properties of a tissue type or microstructures of a tissue type of an animal to be determined by OCT imaging. Suitably the database of optical properties comprises at least one optical property of the tissue selected from the transmission, diffuse reflectance, scattering and absorption characteristics, preferably over a wavelength range of 500 nm to 2200 nm, more preferably over a range of 600 nm and 2000 nm.

Preferably the database is an electronic database, and more preferably stored on an electronic computer, when present. Suitably the electronic computer comprises means to compare optical properties of an OCT image with the optical properties on the database. Preferably the electronic computer comprises means to indicate when a potential match between at least one optical property of an OCT image and the optical properties stored on the database for a specific tissue is determined.

According to a third aspect of the invention, there is provided use of a kit of the second aspect of the invention to perform the method of the first aspect of the invention.

### BRIEF DESCRIPTION OF THE DRAWINGS

For a better understanding of the invention and to show how embodiments of the same may be put into effect, the various aspects of the invention will now be described by way of example only with reference to the accompanying diagrams, in which:

FIG. 1 illustrates a schematic diagram of an optical coherence tomography system useful in any of the aspects of the invention;

FIGS. 2a and 2b show images of healthy human lung tissue, by standard histological section (FIG. 2a) and by optical coherence tomography (FIG. 2b);

FIGS. 3a and 3b show images of inflamed human lung tissue by standard histological section (FIG. 3a) and by optical coherence tomography (FIG. 3b);

FIGS. 4a and 4b show images demonstrating the deposition of carbon pigment in human lung tissue by standard histological section (FIG. 4a) and by optical coherence tomography (FIG. 4b);

FIGS. 5a and 5b show images of cancerous human lung tissue by standard histological section (FIG. 5a) and optical coherence tomography (FIG. 5b);

FIGS. 6a and 6b show images demonstrating metaplastic squamous epithelium in human bronchus by standard histological section (FIG. 6a) and by optical coherence tomography (FIG. 6b).

### DESCRIPTION OF THE PREFERRED EMBODIMENTS

### Examples

### Methods

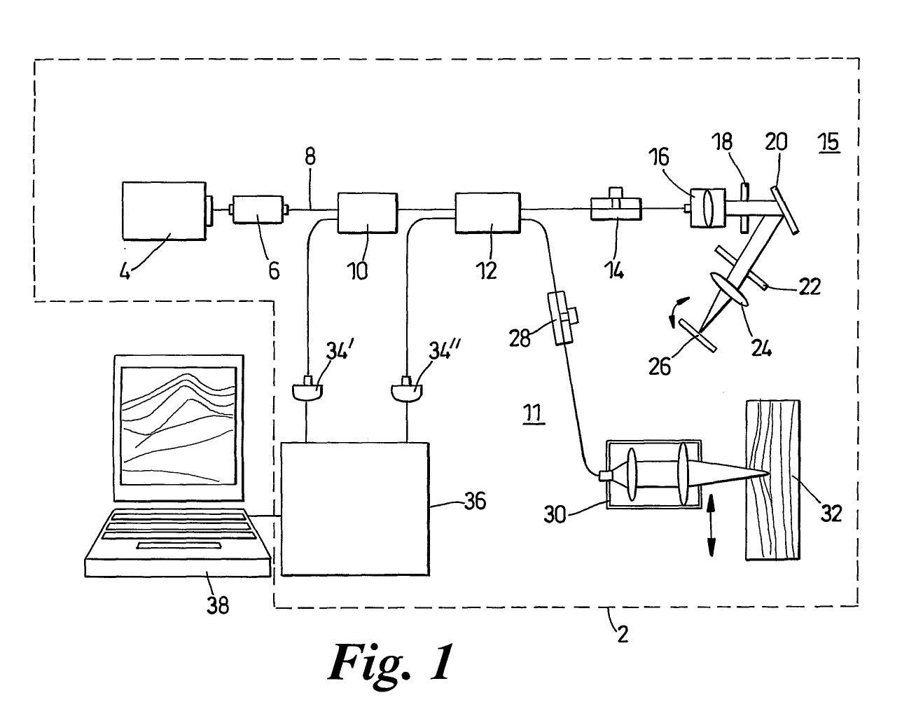

FIG. 1 depicts a schematic diagram of an OCT (optical coherence tomography) system useful in the invention. OCT measures the intensity of backscattering light within a tissue sample by means of interference and uses this to represent the tissue optical discontinuity.

A bench-top optical coherence tomography (OCT) system was built incorporating a Michelson-type interferometer (2) comprising a broad band light source (4), a 1300 nm superluminescent diode with a bandwidth of 52 nm coupled into a single optical fibre (8), and split by a 50/50 optical fibre coupler (10), the coupler (10) being coupled to the light source (4) by way of an isolator (6). The light source (4) used yields a 10 μm axial resolution in lung tissue. 50% of the light is directed to a reference arm (15) of the interferometer (2) where a rapid double-pass scanning system is employed to modulate the interference signal and provide the optical path length scanning. The reference arm comprises a collimator (16) connected to a neutral density filter (18), a grating (20), double pass mirror (22), optical lens (24) and finally a reflecting mirror (26). The residual light is directed towards a test sample (32) by way of a probe head (30) with a fibre optic cable (11) split from the cable (8) by way of a 2×2 coupler (12). The probe head (30) comprises The probe head (30) includes a lens system made up of a collimating lens and an objective lens which focus the infrared beam onto the test sample (32), with focusing optics. A high resolution motorised translation stage (not shown) accurately controls the movement of the mirror (26). Light backscattered from the sample (32) is combined with light reflected from the mirror (26). These beams interfere only if the optical path lengths of the two beams are matched to within the coherence length of the light. Polarisation controllers (28) are used in both arms to achieve the maximum obtainable interference fringe visibility. The system employs a balanced detector scheme (34′, 34″) to minimise the fluctuation noise arising from the light source (4). The light reflected from the sample (32) and mirror (26) is then passed through a differential amplifier, filter and demodulator system (36) before being analysed in a computer (38). The transverse resolution was measured at 16 μm, limited by the numerical aperture of the lens used to deliver the light onto the sample, and the optical frequency of the incident light as in conventional microscopy. The signal-to-noise ratio (SNR) of the system was measured at 100 dB by the use of a 4 OD neutral density filter.

Following informed written consent, lung airway section samples were obtained from 15 patients undergoing total pneumonectomies (3) lobectomies (5 patients) or partial lobectomies (7 patients) for lung cancer and were scanned using the above OCT system prior to histological processing. The samples were kept moist by phosphate buffered saline to avoid dehydration of the samples during the scanning process. The exact location of each scan was marked using a fine needle and thread, which clearly defined the starting point of each image. These markers acted as a guide for the subsequent tissue sampling for microscopy and histopathological staining, ensuring microscopic examination of the same anatomical location to the OCT image. The position of the probe head beam on the scanned tissue was monitored using a visible light guiding beam; and the optical probe head was never in contact with the sample. All the OCT scanning was performed on the luminal surface of the resected airways samples, examining longitudinal sections of each sample sequentially from macroscopically disease-free portions of the samples, to inflamed, pre-cancerous and cancerous states, right up to and including site of tumour in the sample. The scanning area varied from 2×6 mm to 2×12 mm (depth×length). Following OCT scanning, the airway sections samples were fixed in 10% buffered formalin for 48 h and subjected to standard paraffin embedding processing. Sections approximately 5 μm thick were cut from the samples at the marked tissue sites and stained with haematoxylin and eosin to provide a library of histopthological images of the tissue in various stages of a disease state for comparison with the OCT images.

To avoid observer bias, analytical comparison of the recorded tomograms and measurement of structural dimensions were carried out independently by two histopathologists and the inter-observer variability assessed. Intra-observer variability was tested by each observer independently repeating above OCT to histopathology comparisons for all images/sections three times separated by an interval of 8 weeks.

### Results

### Microstructural Characterisation of Normal (Disease-Free) Bronchial Airway.

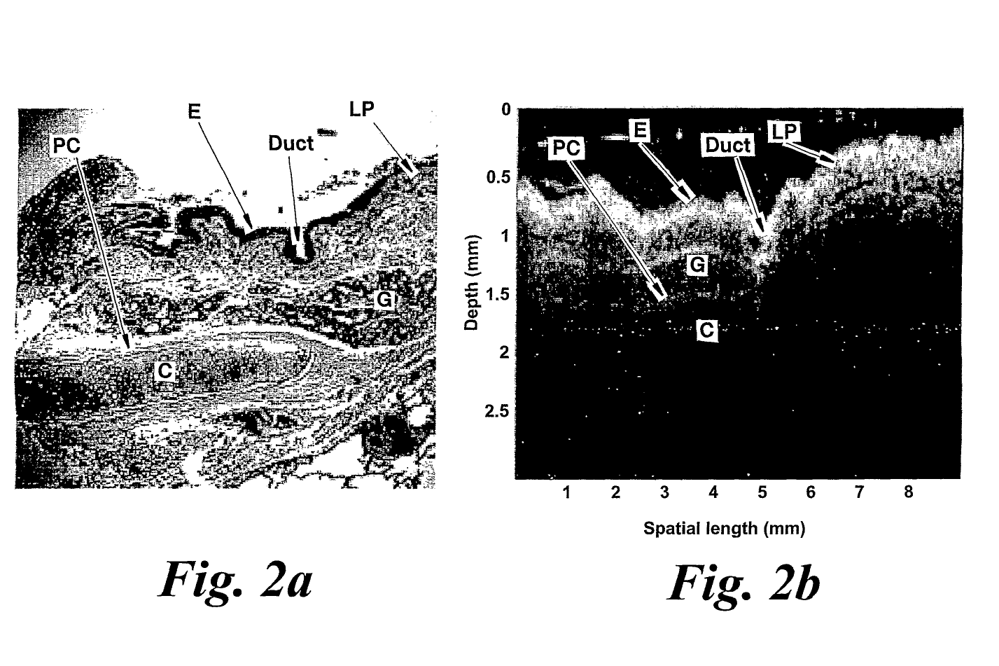

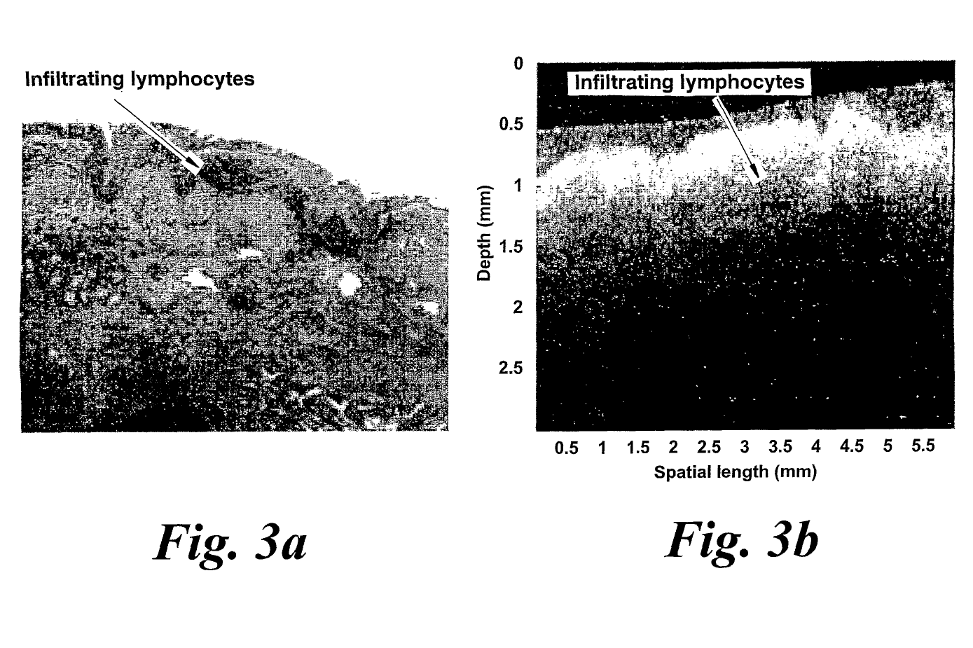

As described above, a corresponding library of histology sections of the scanned macroscopically disease-free and diseased airways (FIGS. 2a, 3a,4a, 5a, 6a) were used for validation of the OCT images. Histological analysis demonstrated that human airways are formed by concentric tissue layers, which varied in thickness, microscopic structure, function and biological behaviour. FIG. 2a shows the histological image of one sample of disease-free bronchial wall and demonstrates the different layers that characterise healthy human airways. The airway is lined by respiratory epithelium which consists of a single layer of ciliated columnar cells resting on a thin layer of basement membrane separating the epithelium from the underlying lamina propria. The lamina propria is a zone of elastin rich connective tissue that forms the deep border of the mucosa and gradually merges with the underlying submucosa. Smooth muscle, mucous glands and outer cartilage plates are all distributed within the submucosa amidst blood vessels and connective tissue. Glands in the submucosa connect with the airway lumen by short ducts opening on the mucosal surface. Cartilage plates keep the airway open and are surrounded by a layer of perichondrial collagen rich connective tissue. Such cartilage is present in the trachea and extra-pulmonary bronchi, becoming smaller and fragmented in the intrapulmonary airways and absent in bronchioles.

The OCT images were able to separate out the component layers which make up the airway wall by detecting the inherent different refractive indices and scattering properties to incident light of the individual composite structural elements. Specifically, in the disease-free human bronchial section sample subsequently used for the histopathological image shown in FIG. 2a, OCT precisely delineated the following anatomical components: epithelium (E), lamina propria (LP), smooth muscle (SM), mucus glands (G) and cartilage (C) as shown in FIG. 2b. The transition between these microstructural layers was well defined and closely mirrored the layered appearance subsequently profiled on the histology image. The demarcation of the epithelium, mucous gland ducts and cartilage is particularly well defined; the lamina propria and submucosal structures are also easily recognisable by OCT. Variation in OCT definition across the different layers of the airway wall can be explained by the presence of a higher nuclear density within structures such as the epithelium and cartilage, reflected in enhanced refractive indices as compared to adjacent surrounding tissues. Thus, the relatively higher refractive index of a particular structure results in sharper OCT image interpretation. For example, the comparatively denser extracellular matrix of cartilage decreases scattering of incident light, and so reflects as a dark region on the OCT tomogram. The connective tissue layer including smooth muscle beneath the epithelium is clearly imaged.

The relative dimensions of structural components are accurately depicted on the OCT images of FIGS. 2b, 3b, 4b, 5b and 6b. The measured thickness on OCT of the epithelium and cartilage were 100±25 μm and 450±15 μm respectively, whilst the intervening distance from epithelium to cartilage was 250±28 μm; as compared to their histological quantification of 84±21 μm, 378±30 μm and 210±42 μm respectively. The relatively small differences may be attributable to expected shrinkage of lung tissue following histological processing.

Across all the sample sections, OCT imaging penetrated the full thickness of the airway wall to at least the outer confines of cartilage plates. Clear imaging was seen to a maximum penetration depth up to 2.5 mm with a spatial resolution of 10 μm and a scanning speed of 1 frame per second. The above findings were consistent across serial sections performed on the 15 resected patient lung samples (20-30 scans for each patient).

### Identification of In Situ Inflammatory and Neoplastic Pathology

OCT can accurately capture the composite airway architecture up to a depth of 2.5 mm. This ‘optical fingerprint’ provides details of changes occurring beneath the epithelial surface by reflecting the morphology of the main airway components invisible to the naked eye. Presence of chronic intense inflammation tends to homogenize tissue and disrupts tissue boundaries as can be seen in FIGS. 3a and 3b.

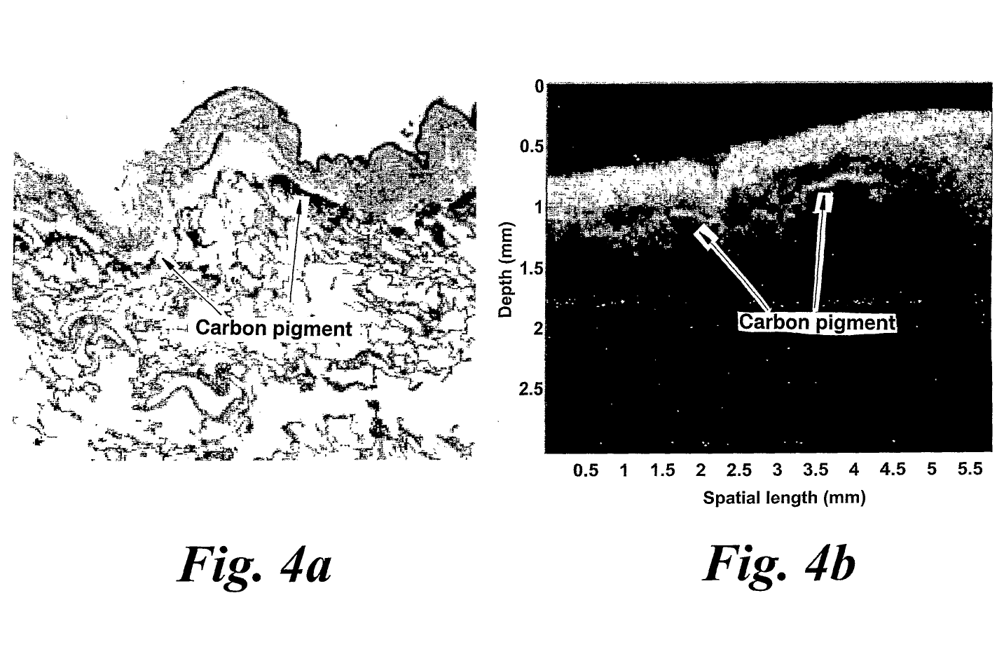

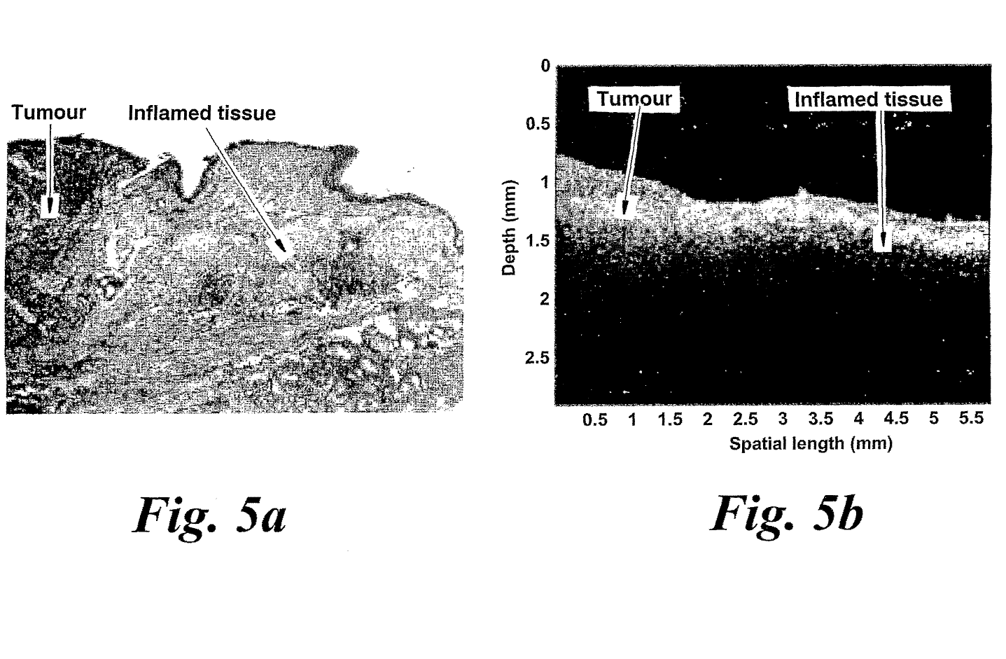

In one sample from a patient known to have had a heavy smoking history and exposure to coal dust, OCT clearly identified deposition of carbon pigment within the bronchial epithelium as shown in FIGS. 4a and 4b. Histologically, granular black carbon pigment, when present, is often visualised along lymphatics. On the OCT tomograms of FIG. 4b the heavy deposition of carbon pigment is reflected as a clearly identifiable separate bright layer within the epithelial microstructural profile. OCT images of airway sections immediately leading to and including site of tumour were compared to histological analysis of same sections. Histologically, tumour presence is characterised by destructive growth ignoring and effacing the normal tissue boundaries. This loss of normal tissue architecture was captured by OCT, which at the present spatial resolution of 10 μm produces a featureless image lacking the ordered multi-layered appearance of the healthy airway wall as can be seen in FIG. 5b. Thus the clear demarcations of epithelium and lamina propria as seen in the samples of inflamed tissue shown in FIG. 3b are lost.

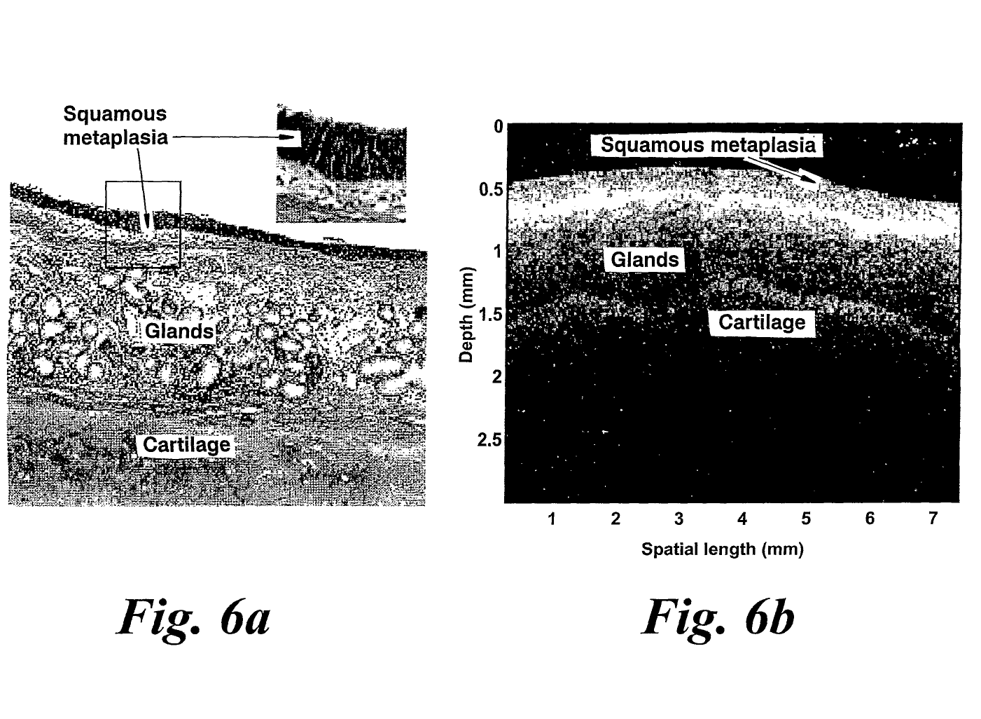

In some samples, detailed analysis of the scanned sections of airways showed histological presence of squamous metaplasia. Replacement of the normal single layer of ciliated respiratory epithelium by multi-layered squamous epithelium often occurs in smokers and provides a suitable environment for early morphological changes associated with lung tumour development. Areas of squamous epithelium are thicker and possess different cellular morphology when compared with the appearance of normal respiratory epithelium by light microscopy and the results shown in FIG. 6a. This increase in the thickness of epithelium which characterises squamous metaplasia was also captured on the corresponding OCT image as shown in FIG. 6b, and contrasts with the images of airways lined by a single layer of normal respiratory epithelium.

The representative OCT images were consistent across numerous lung section samples performed on the 12 patients. The coefficient of variability of the differences between the 3 separate analyses of OCT image to histopathology by both histopathologists was between 3% and 10%; and altogether that between the two was <5%.

Thus, a library of histopathological images (FIGS. 2a-6a) can be utilised as benchmarks in order to compare corresponding OCT images of other samples of tissue, with the OCT images being taken in situ and compared real time to the reference library of histopathological image library.

### Discussion

Real time OCT images of freshly obtained human airways were compared to gold standard histopathological analysis. It was found that OCT imaging is a sensitive optical biopsy device to characterise the highly organised multilayered architecture of the healthy bronchial airways, with excellent histological correlation in terms of structural profiles and dimensions. In addition, it has been shown that OCT is able to identify, in situ, morphological changes associated with inflammation and neoplasia within the airway wall.

OCT utilises light signals rather than sound; delivering infrared light waves to the imaged tissue through a single optical fibre. Light then reflects off the internal structural layers within the scanned section, allowing micron-scale resolution pick-up of normal anatomy and in situ morphological aberrations. The morphology of individual anatomical components varies in relative thickness, cellular composition and density as well as relative amount of acellular extracellular matrix, as revealed by standard histological analysis. This results in inherent different optical properties, such as optical scattering, reflection and transmission. Thus although the contrast mechanism in OCT is different from normal light microscopy, the high sensitivity of OCT enables differentiation of the individual microstructures that make up the airway wall, ensuring comparable images to histological sections.

These results support the capability of OCT to image human airways and detect airway pathology in real time. As more than 85% of lung tumours originate within the bronchial epithelium, with progressive cellular changes developing over a long period prior to discovery of invasive cancer, OCT has been shown to provide critical diagnostic information of early malignant in situ changes.

OCT images can be compared to a library of histopathological images of tissues at various stages of a disease state to enable in situ, determination of a disease state, or determination remote to the patient.

In preferred embodiments the histopathological images, as shown in FIGS. 2A, 3A, 4A, 5A and 6A, for example, are preferably added to an electronic database, as are the OCT images, so that they may be easily compared by electronic means such as an electronic computer 38, which can read the databases of both the histopathological images and OCT images and flag up to a user those images that are the closest match. Match criteria may include the presence or absence of certain tissue structures, morphology of certain tissue structures or a combination thereof, for example.

In particularly preferred embodiments, electronic means to compare the histopathological image database to the OCT image database preferably comprises means to indicate to a user when a potential match between a non-diseased tissue in the histopathological image database matches a target OCT image, when an OCT image potentially matches a corresponding histopathological image of a tissue having a possible disease state and/or when a target OCT image potentially matches a corresponding histopathological image showing a tissue having a definite disease state (e.g. showing a tumour or cancer cells, for example). In this way a user can be alerted when a particular OCT image of a tissue potentially matches a known histopathological image of a tissue in an acute or high risk disease state, such as cancer, for example.

In preferred embodiments, the electronic computer 38 also includes a database of optical properties of the target tissue type, which optical properties may include transmission, diffuse reflectance, scattering and absorption characteristics of the tissues or microstructures within the tissue. Suitably the optical properties are located in a database on the electronic computer. The optical properties may be determined by spectrophotometry, using for example, a Varian Cary 500 spectrophotometer, over a wavelength range of between 500 nm and 2200 nm, more preferably 600 nm to 2000 nm. In particularly preferred embodiments, means to compare optical properties of a target tissue type from an OCT image with the optical properties on the database are present in the electronic computer. Preferably the electronic computer also includes means to indicate to a user when a potential match between optical properties of a tissue or microstructure within a tissue of an OCT image has been matched with optical properties of a specific tissue type on the database.

The reader's attention is directed to all papers and documents which are filed concurrently with or previous to this specification in connection with this application and which are open to public inspection with this specification, and the contents of all such papers and documents are incorporated herein by reference. All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features. The invention is not restricted to the details of the foregoing embodiment(s). The invention extend to any novel one, or any novel combination, of the features disclosed in this specification (including any accompanying claims, abstract and drawings), or to any novel one, or any novel combination, of the steps of any method or process so disclosed.

1. A method of determining a disease state of an animal, comprising the steps of:
(a) providing a plurality of histopathological images of a tissue type of an animal at various stages of a disease state;
(b) imaging a sample of at least a portion of the same tissue type of a target animal by optical coherence tomography scanning; and
(c) comparing the optical coherence tomographical scanned image with the histopathological images of the tissue to determine the disease state of the animal.

2. The method of claim 1, where the step (a) comprises providing histopathological images of a tissue type of an animal at various stages of a cancerous state.

3. The method of claim 2, wherein the step (a) comprises providing histopathological images of a tissue type at least in a non-cancerous, pre-cancerous, inflamed and cancerous state.

4. The method of claim 3, wherein the step (a) comprises providing a plurality of histopathological images of a tissue type for each of the non-cancerous, pre-cancerous, inflamed and cancerous states.

5. The method of claim 4, wherein the step (a) comprises providing a plurality of histopathological images of a tissue type for each of the non-cancerous, pre-cancerous, inflamed and cancerous states, showing variations within specific structures in the tissue type for each state.

6. The method of claim 1, wherein the tissue type is lung tissue.

7. The method of claim 1, wherein the animal is a human.

8. The method of claim 1, wherein imaging of the tissue sample in step (b) by optical coherence tomography comprises optical coherence tomography scanning using a Michelson-type interferometer.

9. The method of claim 1, wherein the step (b) comprises imaging a sample in situ of a tissue type of the target animal by optical coherence tomography scanning.

10. The method of claim 1, wherein the step (b) comprises removing a sample of the tissue from the target animal and imaging at least a portion of the removed tissue remote from the animal.

11. The method of claim 1, wherein the step (c) comprises comparing a single optical coherence tomogram image with one or more of the plurality of histopathological images.

12. The method of claim 11, wherein the step (c) comprises comparing a single optical coherence tomogram image with all of the histopathological images, to determine which of the histopathological images substantially correlates with the optical coherence tomogram.

13. The method of claim 1, wherein the step (c) comprises comparing a plurality of optical coherence tomogram images of the tissue of the target animal with one or more of the plurality of histopathological images.

14. The method of claim 1, wherein the histopathological images are stored in an electronic database.

15. The method of claim 1, wherein the or each optical coherence tomogram image is stored in an electronic database.

16. The method of claim 13, wherein:
the histopathological images are stored in a first electronic database;
the or each optical coherence tomogram image is stored in a second electronic database; and
wherein the step (c) comprises comparison of the databases by electronic computing means, the electronic computing means comprising means to indicate when a potential match occurs between a optical coherence tomogram image and a histopatholical image.

17. The method of claim 1 further comprising the step of generating a database of optical properties of the tissue type or microstructures of the tissue type.

18. The method of claim 17, further comprising comparing the optical coherence tomographical scanned image with the database of optical properties of the tissue, to determine properties of the tissue scanned in the optical coherence tomogram image.

19. The method of claim 17, wherein the database of optical properties is an electronic database associated with an electronic computer, and the method further comprises the steps of: comparing the optical coherence tomographical scanned image with the database of optical properties electronically; and indicating to a user of the electronic computer when a match of one or more optical properties from the database of optical properties of the tissue type occurs with an optical coherence tomogram image.

20. A kit comprising
(a) an optical coherence tomography apparatus comprising an interferometer comprising a broadband light source, a beam splitter, a reference arm comprising a reference mirror, and a sample arm comprising a sample probe through which light emitted from the broadband light source may pass, and a means to analyse light reflected from the reference arm and sample probe;
(b) a library of histopathological images of a tissue type of an animal at various stages of a disease state; and
c) an electronic computer for storing a database of optical coherence tomogram images produced by the interferometer, wherein the electronic computer comprises means to compare an optical coherence tomogram image with histopathological images contained in the library of histopathological images, and means to indicate when a potential match between an optical coherence tomogram image and a histopathological image from the image library occurs and wherein the means to indicate when a potential match occurs is arranged to detect a match based on whether a particular optical coherence tomogram image of a target tissue comprises one or more of the same structures as a histopathological image in the library.

21. The kit of claim 20,
wherein the means to indicate when a potential match occurs is arranged to detect a match based on whether a particular optical coherence tomogram image of a target tissue comprises one or more of the same structures or features from a histopathological image in the library, the structures or features selected from a group consisting of:
homogenization of tissue and disruption of tissue boundaries;
deposition of carbon pigment within the bronchial epithelium;
destructive growth ignoring and effacing normal tissue boundaries;
loss of clear demarcations of epithelium and lamina propria; and
replacement of a normal single layer of ciliated respiratory epithelium by multi-layered squamous epithelium.

22. (canceled)

23. The kit of claim 20 further comprising a database of optical properties of a tissue type or microstructures of a tissue type of an animal determined by optical coherence tomogram imaging.

24. The kit of claim 23, wherein the database of optical properties is an electronic database stored on an electronic computer.

25. The kit of claim 24, wherein the electronic computer comprises means to compare optical properties of an optical coherence tomogram image with the optical properties in the database, and means to indicate when a potential match between at least one optical property of an optical coherence tomogram image and the optical properties stored in the database for a specific tissue is determined.

26. Use of the kit of claim 20 to perform the method of claim 1.
